(19) United States
(12) Patent Application Publication
(10) Pub. No.: US 2026/0263164 A1
(43) Pub. Date: Sep. 10, 2026

(54) THE COLORIZATION OF MEDICAL DEVICES IN ROBOTIC SURGERY USING AI AND MACHINE LEARNING

(71) Applicant: Clear Biopsy LLC, St. Joseph, MO (US)

(72) Inventor: Steven POPLAW, St. Joseph, MO (US)

(21) Appl. No.: 19/660,558

(22) Filed: Apr. 28, 2026

Related U.S. Application Data
(63) Continuation of application No. 19/430,651, filed on Dec. 23, 2025.
Application No. 19/430,651 is a continuation-in-part of application No. 19/307,577, filed on Aug. 22, 2025, now Pat. No. 12,667,439.
(60) Provisional application No. 63/711,850, filed on Oct. 25, 2024.
(60) Provisional application No. 63/898,869, filed on Oct. 14, 2025.

(51) Int. Cl. A61B 34/20; A61B 34/30; A61B 90/00; A61B 90/92

(52) U.S. Cl. CPC A61B 34/20; A61B 34/30; A61B 90/37; A61B 90/92; A61B 2034/2057; A61B 2034/2065; A61B 2034/301; A61B 2090/378

(57) ABSTRACT

A system for intraoperative medical instrument recognition may receive, from an imaging device positioned inside of a patient, a stream of intraoperative three-dimensional (3D) imaging data that includes anatomy of a patient and a medical instrument. The system may apply a material recognition algorithm to identify one or more objects formed of a predetermined material present in the intraoperative 3D imaging data. The system may apply a shape recognition algorithm to the one or more identified objects to identify the medical instrument. The system may generate a modified intraoperative imaging data stream in real-time with the receiving of the stream of intraoperative 3D imaging data. The modified intraoperative imaging data stream may include a visual characteristic, e.g., a colorization, applied to a region of the intraoperative 3D imaging data corresponding to the at least one identified medical instrument.

### RELATED APPLICATIONS

This application claims priority to U.S. Provisional Application No. 63/711,850, filed Oct. 25, 2024, U.S. Provisional Application No. 63/898,869, filed Oct. 14, 2025, U.S. Non-Provisional application Ser. No. 19/307,577, filed Aug. 22, 2025, and U.S. Non-Provisional application Ser. No. 19/430,651, filed Dec. 23, 2025, the entireties of which are incorporated herein.

### TECHNICAL FIELD

Various embodiments of this disclosure relate generally to machine-learning-based techniques for object recognition during medical procedures, and, more particularly, to systems and methods for identifying one or more medical devices in a surgical imaging scene and determining tool states thereof.

### BACKGROUND

Medical procedures are often performed inside the body where the target and/or the instrument are hidden from the naked eye. Medical imaging is often used to provide imaging inside the body before, during or after such procedures, but it is often difficult to properly appreciate details in the imaging. In fact, depending on the circumstances, even seasoned professionals may improperly glean certain shadows or tones in the conventional imaging. Further, it remains difficult to ascertain certain imaging features, such as metal components and/or medical instruments. This may present patient safety issues and lead to injury or death. It may also prolong the length of a procedure as the physician or operator struggles to properly position instruments. The quality of the imaging may also lead to missing suspicious lesions or yielding false negative biopsies.

Conventional techniques, including the foregoing, fail to recognize, emphasize, or otherwise highlight certain objects routinely featured during medical imaging procedures, including, but not limited to, bodily features and medical instruments, and portions thereof. This disclosure is directed to addressing challenges such as one or more of those referenced above. The background description provided herein is for the purpose of generally presenting the context of the disclosure. Unless otherwise indicated herein, the materials described in this section are not prior art in this application and are not admitted to be prior art, or suggestions of the prior art, by inclusion in this section.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate various exemplary embodiments and together with the description, serve to explain the principles of the disclosed embodiments.

FIG. 1 depicts an exemplary environment for intraoperative medical instrument recognition, according to aspects of the present disclosure.

FIGS. 2A-2C are photographs showing a first target site without and with annotation, according to aspects of the present disclosure.

FIGS. 3A-3B are photographs showing a first target site without and with annotation, according to aspects of the present disclosure

FIG. 4 is a flowchart illustrating an exemplary method for intraoperative medical instrument recognition, according to aspects of the present disclosure.

FIG. 5 is a flowchart illustrating an exemplary method for intraoperative medical instrument recognition with dynamic tool state determination, according to aspects of the present disclosure.

FIG. 6 is a flowchart illustrating an exemplary method for object recognition during a medical procedure with grip force monitoring and over-grip condition detection, according to aspects of the present disclosure.

FIG. 7 depicts a perspective view of a robotic surgery device with an integrated actuator sensor, according to aspects of the present disclosure.

### SUMMARY

In some aspects, the techniques described herein relate to a computer-implemented method for intraoperative medical instrument recognition, the computer-implemented method including: receiving, from an imaging device positioned inside of a patient, a stream of intraoperative three-dimensional (3D) imaging data that includes anatomy of a patient and at least one medical instrument; applying a material recognition algorithm to identify one or more objects formed of a predetermined material present in the intraoperative 3D imaging data; applying a shape recognition algorithm to the one or more identified objects to identify the at least one medical instrument; determining, using a dynamic tool state algorithm, based on one or more of the stream or tool data received from the at least one medical instrument, a tool state of the at least one medical instrument; and generating a modified intraoperative imaging data stream in real-time with the receiving of the stream of intraoperative 3D imaging data, wherein the modified intraoperative imaging data stream includes a visual characteristic applied to a region of the intraoperative 3D imaging data corresponding to the at least one identified medical instrument, wherein the visual characteristic may be indicative of the determined tool state.

In some aspects, the techniques described herein relate to a computer-implemented method for object recognition during a medical procedure, including: receiving, from an imaging device positioned inside of a patient, an imaging scene of a target, the imaging scene including a medical instrument; applying a metal recognition algorithm to identify metal present in the imaging scene; applying an object recognition algorithm to the identified metal to identify the medical instrument; applying a machine-learning model, including a tool state recognition algorithm to the medical instrument to determine, based on one or more of a stream of intraoperative three-dimensional (3D) imaging data or tool data received from the medical instrument, a tool state of the medical instrument; and generating a modified imaging scene that includes a visual characteristic applied to the identified medical instrument, wherein the visual characteristic may be indicative of the tool state.

In some aspects, the techniques described herein relate to a system for intraoperative medical instrument recognition, including: at least one medical instrument; that may include at least one actuator sensor configured to generate tool data indicative of a tool state of the at least one medical instrument; at least one imaging device configured to capture intraoperative three-dimensional (3D) imaging data; and an imaging analysis device that includes: at least one memory storing: instructions for intraoperative medical instrument recognition; a first machine-learning model that has been trained to identify at least one material included in the at least one medical instrument based on input imaging data, and to segment or generate a reconstruction of a shape of the identified at least one material in the imaging data; and a second machine-learning device that has been trained to recognize the at least one medical instrument based on an input shape; and a dynamic tool state recognition algorithm configured to recognize tool states based on one or more of the imaging data or tool data from the at least one actuator sensor; and at least one processor operatively connected to the at least one memory and configured to execute the instructions to perform operations including: receiving, from the imaging device, a stream of intraoperative 3D imaging data that includes anatomy of a patient and at least one medical instrument at least partially inserted into the anatomy; applying the first machine-learning model to the intraoperative 3D imaging data to identify one or more regions of the intraoperative 3D imaging data that include the at least one material, and to segment or generate a shape of the at least one material; applying the second machine-learning model to the shape to identify the at least one medical instrument; applying the dynamic tool state algorithm to the at least one identified medical instrument to identify a tool state of the at least one medical instrument; and generating a modified intraoperative imaging data stream in real-time with the receiving of the stream of intraoperative 3D imaging data, wherein the modified intraoperative imaging data stream includes a visual characteristic applied to a region of the intraoperative 3D imaging data corresponding to the at least one identified medical instrument, wherein the visual characteristic may be indicative of the identified tool state.

### DETAILED DESCRIPTION OF EMBODIMENTS

According to certain aspects of the disclosure, methods and systems are disclosed for object analysis and recognition during medical procedures, e.g. surgical imaging (including videos and images). It remains difficult to ascertain certain imaging features, such as metal components and/or medical instruments. This may present patient safety issues and lead to injury or death. However, conventional techniques may not be suitable. For example, conventional techniques may not adequately identify and indicate (or otherwise highlight and/or emphasize) certain objects/elements (and characteristics thereof) within an imaging scene. Accordingly, improvements in technology relating to object analysis, object recognition, and corresponding user interface elements are needed.

The systems, devices, and methods may apply artificial intelligence and/or machine learning techniques to enhance medical imaging object recognition. The exemplary embodiments may be used by a system to perceive one or more physical characteristics of surgical devices from images, video, and other media (including 3D formats). The systems, devices, and methods of the disclosure may be applied preoperatively to existing media (e.g., for training or review purposes), intraoperatively (e.g., to assist during a medical procedure), and/or post-operatively (e.g., for training or review purposes). The systems, devices, and methods may be used by humans and/or robotic surgical systems. In an example, a robotic surgical system may apply object recognition techniques described herein to improve its own surgical capabilities.

As will be discussed in more detail below, in various embodiments, systems and methods are described for using machine learning to improve object recognition during medical procedures. By training a machine-learning model, e.g., via supervised or semi-supervised learning, to learn associations between training data and ground truth data, the trained machine-learning model may be usable to identify and analyze objects in a medical imaging scene. It should be understood that the term “scene” as used herein may refer to a given field of view of a medical imaging device, such as a camera probe. Reference to an object being in a given scene should therefore be understood to mean that the field of view of the medical imaging device includes at least a portion of the given object.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially,” “approximately,” “about,” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration. By virtue of such training, a machine-learning model is converted from an un-trained and un-specific model to a model that is unique to and specifically configured for the particular purpose for which it is trained. In an example, training of a machine-learning model is analogous to a method of production in which the article produced is the trained model having unique characteristics by virtue of its particular training. Moreover, the result of training a machine-learning model using particular training data and for a particular purpose results in a technical solution to an inherently technical problem.

The execution of the machine-learning model may include deployment of one or more machine learning techniques, such as linear regression, logistical regression, random forest, gradient boosted machine (GBM), deep learning, or a deep neural network. Supervised or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

In an exemplary use case, a trained machine model may be used by the exemplary systems, devices, and methods disclosed herein to identify and analyze one or more medical imaging scenes. During (or after) a medical procedure, an object recognition algorithm may be used to identify one or more objects in a medical imaging scene. Such identification may be used for various purposes. In one example, the identification may be used to augment a display of the one or more identified objects on an adjustable graphic user interface (GUI). In another example, the identification may be used to guide or augment the operation of a robotic surgery device, e.g., by locating an implement wielded by the surgery device within the body of a patient, by locating anatomy, or the like.

In another exemplary use case, a machine-learning model may be trained to identify one or more characteristics of an identified object. For example, an object recognition algorithm of the systems, devices, and methods of the disclosure may identify that a given object in a scene is a medical instrument. The algorithm may further identify that the given object is of a certain material (e.g., metal), is of a certain size (e.g., dimensional measurements), how or how much of an object is occluded from view, and/or whether the object is of a certain color. In some embodiments, the algorithm may be used to determine a tool state of the medical instrument. For example, the tool state may be indicative of one or more of an open state, a closed state, or a clamped state. In some embodiments, the algorithm may determine a characteristic of an object based on data from an additional source, such as a tool state signal from a sensor or actuator associated with the medical instrument. Any recognized object and characteristic(s) thereof may be displayed on a GUI that may be adjustable by a user. Further description of the GUI is provided below.

While several of the examples above involve medical imaging, it should be understood that techniques according to this disclosure may be adapted to any suitable type of imaging. It should also be understood that the examples above are illustrative only. The techniques and technologies of this disclosure may be adapted to any suitable activity.

Presented below are various aspects of machine learning techniques that may be adapted to recognize, identify, and/or characterize one or more objects in a medical imaging scene. As will be discussed in more detail below, machine learning techniques adapted to medical imaging may include one or more aspects according to this disclosure, e.g., a particular selection of training data, a particular training process for the machine-learning model, operation of a particular device suitable for use with the trained machine-learning model, operation of the machine-learning model in conjunction with particular data, modification of such particular data by the machine-learning model, etc., or other aspects that may be apparent to one of ordinary skill in the art based on this disclosure.

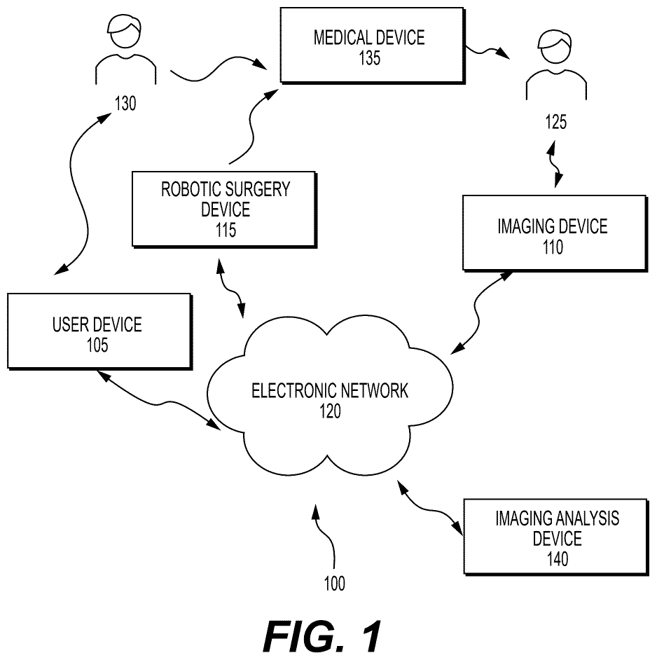

FIG. 1 depicts an exemplary environment 100 according to one or more aspects of this disclosure. The environment 100 may include, for example, a user device 105, an imaging device 110, a robotic surgery device 115, which may communicate via an electronic network 120. A patient 125 may be the focus of a medical procedure associated with a provider 130. The medical procedure may include introduction of one or more medical devices 135 into the body of the patient 125. As discussed in further detail below, an imaging analysis device 140 may be configured to augment medical imaging data generated by the imaging device 110, e.g., by identifying the one or more medical devices 135, anatomy of the patient 125, and/or their relative location or other context.

The user device 105 may include a computer system such as a desktop computer, laptop computer, tablet computer, mobile phone, etc. The user device 105 may include software and/or hardware configured to communicate with or operate in conjunction with other elements of the environment 100. For example, the user device 105 may be configured to operate the robotic surgery device 115, display medical imaging from the imaging device 110, etc.

The imaging device 110 may be configured to capture any suitable type of medical imaging. In an exemplary embodiment, the imaging device 110 may include a Three-Dimensional (3D) video device, a 3D ultrasound device, or any other suitable type of 3D imaging device. The imaging device 110 may be configured to store imaging data in a memory, e.g., of the user device 105, a remote data storage, a cloud storage, or the like. In some embodiments, an imaging device 110 is integrated into a medical device 135. For example, an endoscope may be fitted with a camera or an ultrasound probe, or the like.

The robotic surgery device 115 may include one or more articulatable or robotically controlled arms or digits which may include or be fitted with one or more medical instruments. In some embodiments, the medical devices 135 are medical instruments integrated into or fitted onto the robotic surgery device 115. Examples of such medical instruments include, but are not limited to, graspers, scissors, needle holders or manipulators, suction or irrigation devices, drapes, endoscopes, medical imaging devices, clip appliers, energy devices (e.g., for powering another device such as a laser, cutter, etc.), a cauterizing device, a retractor, a bipolar or laser device, an EndoWrist®, etc. In various embodiments, the robotic surgery device 115 may be controllable, e.g., via the user device 105, and/or may be configured to execute preprogrammed operations.

In some embodiments, the robotic surgery device 115 may be configured to process medical imaging data, e.g., from the imaging device 110, in order to locate a medical device 135 and/or anatomy of the patient 125 and/or other context of a procedure. However, as discussed above, the capability of the robotic surgery device 115 to process medical imaging, like the capability of the provider 130 may be impacted by the difficulty of visualizing or detecting medical instruments in conventional medical imaging. According to one or more aspects of this disclosure, the augmented medical imaging provided by the imaging analysis device 140 may improve the efficiency, accuracy, or speed of the robotic surgery device 115. In some embodiments, however, a robotic surgery device may not be used. For example, the provider 130 may directly manipulate a medical device 135 within the body of the patient 125.

The electronic network 120 may be wired, wireless, or a combination thereof. Such network may be a local or personal network, or may include a connection via the internet. In some embodiments, the electronic network 120 may include or be in communication with an Electronic Medical System (EMS), e.g., a data system at a hospital or the like used to store and communicate patient data and the like.

In some embodiments, other sensors (not shown) may be used to monitor various characteristics of the patient 125, e.g., blood pressure, temperature, neural activity, etc., In some embodiments, such data may be fed to the user device 105, the robotic surgery device 115, the imaging analysis device 140, or the like, which may use such data as additional input when processing imaging data and or guiding use of or operating a medical device 135.

As noted above, in some embodiments, the medical device 135 may be integrated into or affixed onto the robotic surgery device 115. In some embodiments, the robotic surgery device 115, e.g., via use of such a medical device, is operated to manipulate a further medical device. For example, a needle holder may be used to hold or manipulate a needle. Other examples of medical devices 135 include, but are not limited to, a needle assembly, tubular members, needles, trocars, cutting styli, styli, cannula, and/or other components configured to access and sever a tissue sample in a medical procedure commonly referred to as Core Needle Biopsy. However, the foregoing examples are exemplary only, and any suitable medical devices 135 may be used. In some embodiments, the medical device 135 may include at least one actuator sensor 820 configured to generate tool data indicative of a tool state of the medical device 135. The tool data may be based on a signal from the at least one actuator sensor 820. It should be understood that, in various embodiments, any suitable type of sensor for detecting a characteristic or state of a medical instrument may be used. Characteristics that may be sensed include, for example, open/closed state, grip force, orientation, position, motion, contact, fill status, power status, operation time, etc.

As discussed in further detail below, the imaging analysis device 140 may include one or more models or algorithms usable to process imaging data and apply a visual characteristic to medical devices 135 identified therein. In an example, the imaging analysis device 140 may include one or more trained machine-learning models. In an embodiment, a first machine-learning model may have been trained to identify matter in medical imaging that is formed from a particular material, e.g., metal. Further, such model may be configured to segment the identified material, e.g., determine shape or geometry information for the identified material. A second machine-learning model may have been trained to recognize one or more medical devices given a shape or geometry, such as the shape or geometry determined by the first machine-learning model. In some embodiments, the imaging analysis device 140 may include a dynamic tool state recognition algorithm configured to recognize tool states based on one or more of a stream of imaging data or tool data from the at least one actuator sensor 820.

In some embodiments, the dynamic tool state algorithm may be configured to determine a grip force based on tool data, compare the grip force to a predetermined threshold, and in response to the grip force being above the predetermined threshold, generate an output indicative of an over-grip condition of the medical instrument.

In embodiments, the first and/or second machine-learning models may be trained based at least in part on imaging of medical devices 135 within anatomy of one or more patients. In some embodiments, the first and/or second machine-learning models may be trained on a stream or sequence of imaging frames or states. Such training may facilitate identification and recognition operations when a medical device is moving, is partially occluded, is changing shape during operation, or is interacting with anatomy. The dynamic tool state algorithm may be trained based on a training stream or training tool data from at least one actuator sensor 820 and medical instrument labels assigned to one or more of portions of the training stream or the training data from the at least one actuator sensor 820, to predict a likelihood that a particular signal from the at least one actuator sensor corresponds to a particular tool state.

As discussed in further detail below, the imaging analysis device 140 may perform one or more of generating, storing, training, or using a machine-learning model configured to recognize and identify objects in a medical imaging scene. The imaging analysis device 140 may include a machine-learning model or instructions associated with the machine-learning model, e.g., instructions for generating a machine-learning model, training the machine-learning model, using the machine-learning model etc. The imaging analysis device 140 may include instructions for retrieving imaging data, adjusting imaging data, e.g., based on the output of the machine-learning model, or operating the user device 105 to output modified imaging data, e.g., as adjusted based on the machine-learning model. The imaging analysis device 140 may include training data, e.g., teaching data, and may include ground truth, e.g., evaluative data.

In some embodiments, a system or device other than imaging analysis device 140 is used to generate or train the machine-learning model. For example, such a system may include instructions for generating the machine-learning model, the training data and ground truth, or instructions for training the machine-learning model. A resulting trained-machine-learning model may then be provided to imaging analysis device 140.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable. In unsupervised learning, patterns, correlations, or clusters of input samples may be used to determine one or more metrics or features of the samples usable to differentiate between related subsets of the samples. In semi-supervised learning, unsupervised and supervised approaches may be combined.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training or used to validate the trained machine-learning model, e.g., compare the output of the trained model with the ground truth for that portion of the training data to evaluate an accuracy of the trained model. The training of the machine-learning model may be configured to cause the machine-learning model to learn associations between training data and ground truth data, such that the trained machine-learning model is configured to determine an output (e.g., an identified object in a medical imaging scene) in response to the input medical imaging data based on the learned associations. Particular selection or application of training data, such as discussed in various embodiments of this disclosure, may inhibit or reduce impact of concerns such as biasing (e.g., via selection, truncation, or the like), overfitting, under-fitting, etc.

In some instances, training using one set or type of data may be used or adapted to another set of data. For example, a modal initially trained on one data set may require less samples or time to train on a second data set. In another example, initial training may result in a base model that may be tuned with an additional data set so as to form a particularized model specific to circumstances of the additional data set.

In various embodiments, the variables of a machine-learning model may be interrelated in any suitable arrangement in order to generate the output. For example, in some embodiments, the machine-learning model may include image-processing architecture that is configured to identify, isolate, or extract features, geometry, and or structure in one or more of the medical imaging data or the non-optical in vivo image data. For example, the machine-learning model may include one or more convolutional neural network (“CNN”) configured to identify features in the medical imaging data, and may include further architecture, e.g., a connected layer, neural network, etc., configured to determine a relationship between the identified features in order to determine a label and/or characteristic of the identified object.

In some instances, different samples of training data or input data may not be independent. Thus, in some embodiments, the machine-learning model may be configured to account for or determine relationships between multiple samples.

For example, in some embodiments, the machine-learning model of the imaging analysis device 140 may include a Recurrent Neural Network (“RNN”). Generally, RNNs are a class of feed-forward neural networks that may be well adapted to processing a sequence of inputs. In some embodiments, the machine-learning model may include a Long Short Term Memory (“LSTM”) model or Sequence to Sequence (“Seq2Seq”) model. An LSTM model may be configured to generate an output from a sample that takes at least some previous samples or outputs into account. A Seq2Seq model may be configured to, for example, receive a sequence of optical in vivo images as input, and generate a sequence of labels and/or characteristics, in the medical imaging data as output.

Various features may be included or used with any suitable machine learning model. For instance, a model may be configured to receive and or determine a relative positioning of data or portions of data in samples (e.g., location of pixels in an image, etc.), and use such positions as a portion of the input to the model. In another instance, a model configured to utilize attention may be configured to weigh, determine, or the like how different samples or portions of samples impact the output of the model, and may incorporate such data into the training process. An example of a model that utilizes information on relative positioning and attention is a transformer model. One implementation incorporating a transformer is a large language model. Transformers and other suitable models have been used for multi-modal input, e.g., a model that is configured to use and process input of different modalities (a combination of or selection from one or more of text, audio, video, structured or unstructured data, etc.).

Any suitable type of machine learning model or combination of machine learning models may be used. Operations conducted by one model in some embodiments may be distributed amongst a plurality of models in other embodiments, or vice versa.

Certain elements of the environment 100 may have been referred to as distinct devices. However, it should be understood that, in various embodiments, various elements may have one or more components distributed over one or more devices or in one or more locations. In an example, a user device 105 may include a client device proximal to the patient 125 and a server device at a remote location.

In the following systems, devices, and methods, various acts may be described as performed or executed by a component from FIG. 1, such as the imaging analysis device 140 or components thereof. However, it should be understood that in various embodiments, various components of the environment 100 discussed above may execute instructions or perform acts including the acts discussed below. An act performed by a system or device may be considered to be performed by a processor, actuator, or the like associated with that system or device. Further, it should be understood that in various embodiments, various steps may be added, omitted, or rearranged in any suitable manner.

In an exemplary use case, a provider 130 may seek to perform a procedure on a patient 125. The imaging device 110 may be used prior to the procedure, e.g., for planning or review purposes, as well as during the procedure to facilitate the manipulation of one or more medical devices 135.

For example, during a procedure, the imaging device 110 may be capturing imaging data of the anatomy of the patient, e.g., 3D ultrasound imaging. In various examples, a medical device 135 may be advanced to a location within the body through the skin of the patient 125 (percutaneous access), through an open incision or through a body lumen or other structure, a portion of the medical device 135 may be advanced into a lesion or target tissue, or a portion of the medical device 135 may be advanced into the lesion or target tissue to sever a tissue sample from the lesion or target tissue. In some examples, the medical device 135 may be manipulated by the provider 130. In some examples, the medical device 135 may be manipulated by the robotic surgery device 115.

During the procedure, the imaging device 110 may be capturing imaging data of the anatomy of the patient, e.g., a scene that includes at least a portion of the medical device 135. In an example, the imaging device 110 may include an imaging probe inserted into the body of the patient alongside or as part of the medical device 135. In another example, the imaging device may be operated externally to the body of the patient. Any suitable type or combination of types of imaging devices may be used.

Imaging data captured by the imaging device 110 may be fed to the imaging analysis device 140, e.g., as a data stream or the like. The imaging analysis device 140 may process the imaging data, e.g., via one or more machine-learning models, in order to identify the medical device 135 and apply a visual characteristic to it. In an example, the visual characteristic may include a colorization. For instance, different medical devices may have predetermined associations with different colorings, and so a particular coloring may be applied to the portion of a display of the imaging device corresponding the identified medical device 135.

In some embodiments, the object recognition by the imaging analysis device 140 may include the use of multiple machine-learning models. For instance, a first model may have been trained to recognize one or more different materials. Generally, medical devices to be inserted into the body of a patient are formed from biocompatible materials that are distinguishable from body tissue under various types of medical imaging. In optical video, for example, metal generally has a shiny or reflective appearance. In ultrasound imaging, for example, different materials have different echogenic responses based on their density and acoustic properties. The first model may have been trained based on labeled imaging data of different materials viewed under one or more imaging modalities, e.g., in situ within anatomy of a body. Thus, the first model may be trained to identify portions of a scene that include a particular material.

In some instances, the first model may identify a shape of identified material. For example, the first model may identify multiple pixels or voxels that are likely to include a certain material, and then may perform a segmentation process or the like to determine a shape of an object that includes those pixels or voxels. The identified shape may be two-dimensional or three-dimensional. In some cases, two-dimensional imaging data may be usable via the first model to predict or extrapolate a three-dimensional shape of an object. For instance, the first model may be trained using predetermined shapes within various anatomy, and thus may have learned to predict a three-dimensional shape of an object given the context of surrounding anatomy. In some cases, the imaging data may be 3D imaging data, whereby a 3D shape may be determined via segmentation or the like directly.

A second model may be used to recognize which medical device an identified shape corresponds to. For example, the second model may have been trained based on training shape data labeled with associated medical devices. In some embodiments, the first model may be used to generate training data for the second. For example, the first model may be used to generate shape data for known medical devices in various positions and in various contexts, whereby such shaped data may be used along with labels regarding the known medical devices to train the second model.

Such identification and recognition may occur continuously during the procedure. Further, the application of the visual characteristic, e.g., the colorization, may be continuously updated, such that the medical device 135 is colored as it moves or is reoriented, e.g., even if a portion is occluded or moves out of the scene. The visual characteristic may be updated at least at 30 frames per second. In some embodiments, the colorization may be configured to change based on changes in an output of the dynamic tool state algorithm. In some embodiments, a change in the colorization may be proportional to a detected change in the tool state. In some instances, the recognition via the second model includes determination of an orientation or path of motion of the medical device 135. For example, by accounting for the position or motion of the recognized medical device 135 over time (e.g., across frames of the imaging data), the second model may be configured to account for occlusions or changes in perspective of the medical device 135.

In some embodiments, the generating of the modified intraoperative imaging data stream may be based on the one or more of the tool data or the stream of intraoperative 3D imaging data over a period of time, such that the generating includes predicting one or more future position, orientation, motion, or tool state of the at least one medical instrument or a position, motion, orientation, or tool state of an occluded portion of the at least one medical instrument, and the region where the visual characteristic is applied may be based on the predicting.

Further, such operations may be performed for multiple medical devices 135 in the scene, e.g., such that medical devices that might otherwise be hard to distinguish from each other may be clearly disambiguated visually.

In some instances, the modified imaging data, e.g., that includes colorizations, may be output on the user device 105. This may enable a provider 130 to clearly apprehend the state and position of such devices within the body of the patient 125. In some instances, the modified imaging data is fed to the robotic surgery device 115, which may be configured to use the applied visual characteristic to determine the position, orientation, or status of the medical device 135 relative to the anatomy of the patient 125.

The visual characteristics may enable the provider 130 and/or the robotic surgery device 115 to more accurately apprehend the position and state of the medical device 135 and the anatomy of the patient 125, and thus may enable a more accurate and faster completion of the procedure, which may improve patient outcomes. In an example where the procedure includes performing a biopsy, the imaging analysis device 140 may enable more accurate engagement with a biopsy site, leading to a reduction in inaccurate biopsy sampling.

Once the procedure is completed, the medical device 135 may then be withdrawn from the patient 125 and, for example, a tissue sample extracted from a needle assembly may be taken for analysis.

In some embodiments, the computer-implemented methods and systems described herein may include receiving a stream of intraoperative three-dimensional (3D) imaging data from an imaging device 110 positioned inside a patient 125, where the stream includes anatomy of the patient 125 and at least one medical device 135. The methods may further include applying a material recognition algorithm to identify objects formed of a predetermined material, such as metal, present in the imaging data, and applying a shape recognition algorithm to the identified objects to identify the medical device 135. A dynamic tool state algorithm may be applied to determine a tool state of the medical device 135 based on the stream of imaging data or tool data received from actuator sensors 820 within the medical device 135. The tool state may be indicative of an open state, a closed state, or a clamped state. A modified intraoperative imaging data stream may be generated in real-time, including a visual characteristic, such as a colorization, applied to a region corresponding to the identified medical device 135, where the visual characteristic is indicative of the determined tool state. The visual characteristic may be updated at least at 30 frames per second and may change dynamically based on changes in the tool state, with changes in colorization being proportional to detected changes in the tool state.

In some embodiments, the dynamic tool state algorithm may be configured to determine a grip force based on tool data from actuator sensors 820, compare the grip force to a predetermined threshold, and generate an output indicative of an over-grip condition when the grip force exceeds the threshold. The dynamic tool state algorithm may have been trained based on a training stream or training tool data from training actuator sensors and medical instrument labels assigned to portions of the training data, to predict a likelihood that a particular signal from the actuator sensor 820 corresponds to a particular tool state. The generating of the modified imaging data stream may be based on the tool data or the stream of imaging data over a period of time, such that the generating includes predicting future position, orientation, motion, or tool state of the medical device 135, or predicting characteristics of an occluded portion of the medical device 135. The modified imaging data stream may be transmitted to the robotic surgery device 115 configured to manipulate the medical device 135 based on position, orientation, motion, or force indicated by the visual characteristic, or may be displayed on the user device 105 for viewing by the provider 130. The machine-learning model may be configured to use the stream or tool data to auto calibrate in real-time, improving accuracy and adaptability during medical procedures.

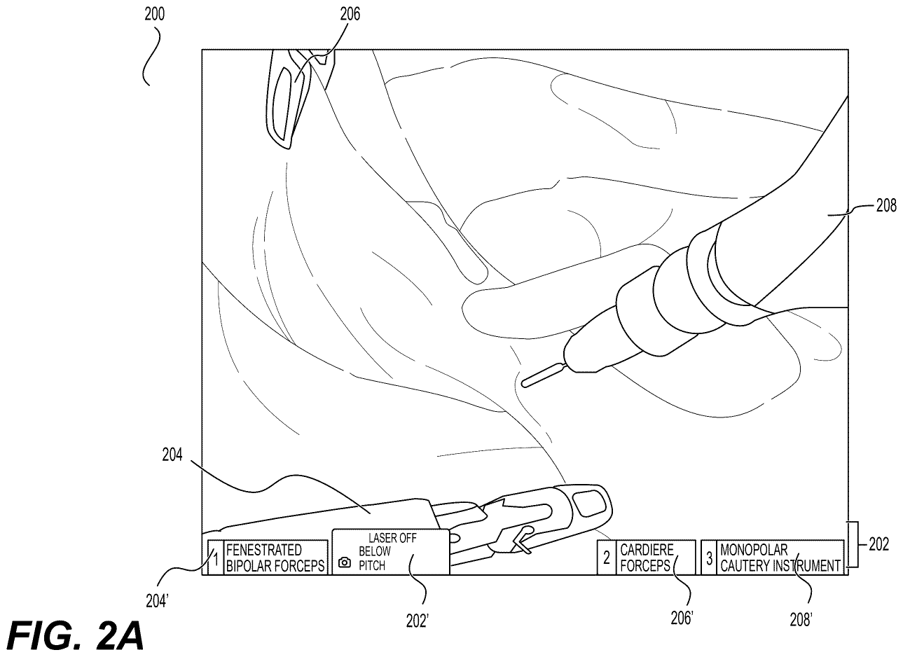

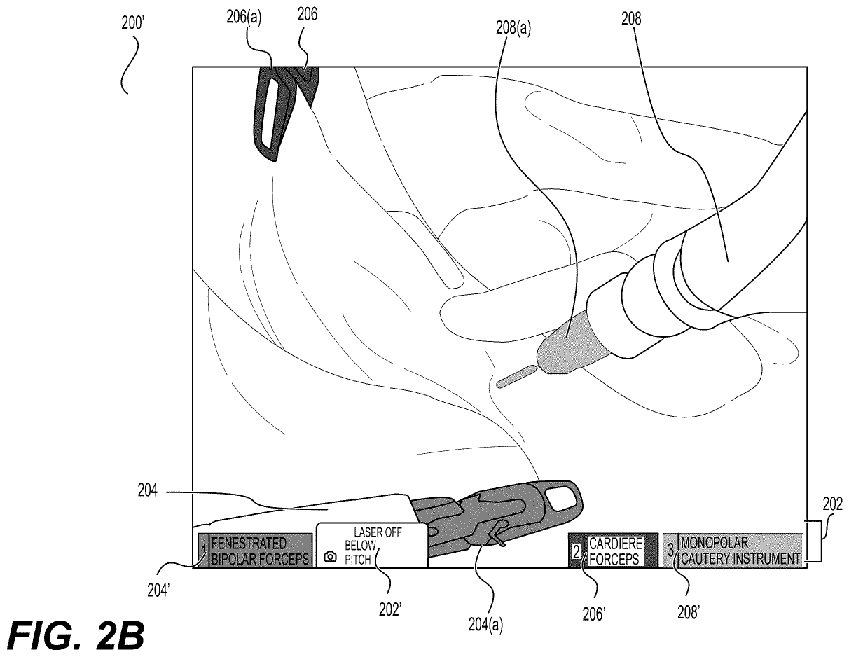

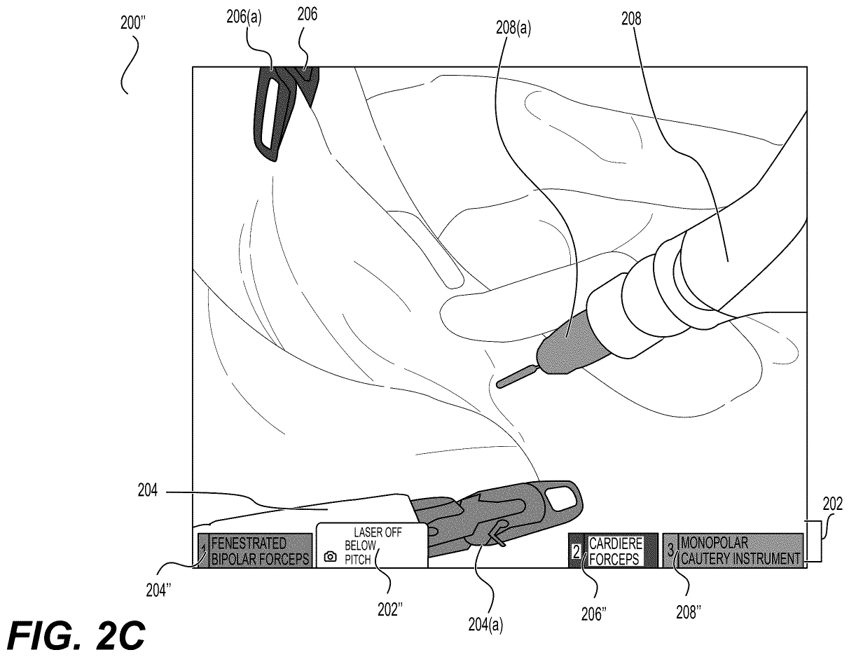

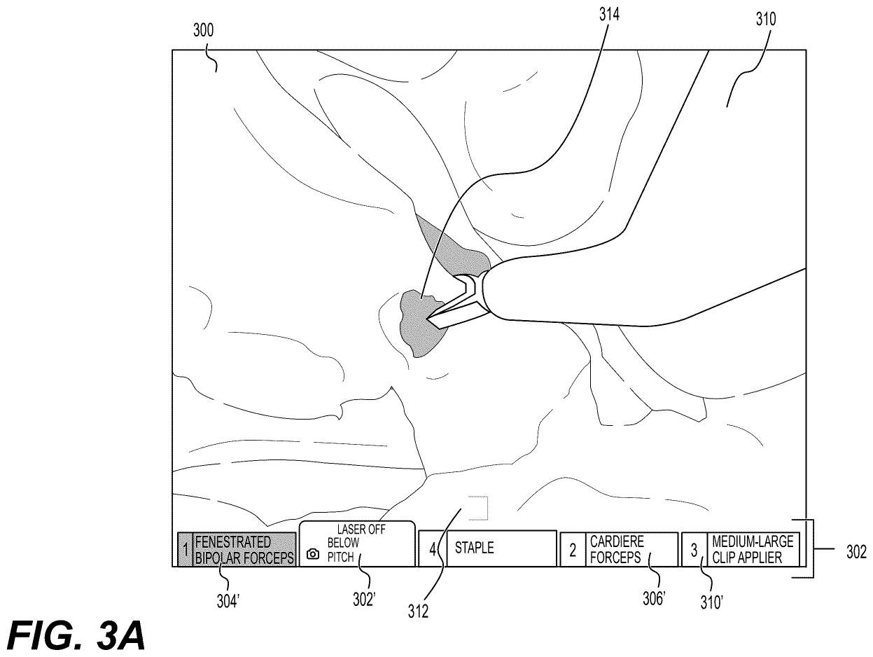

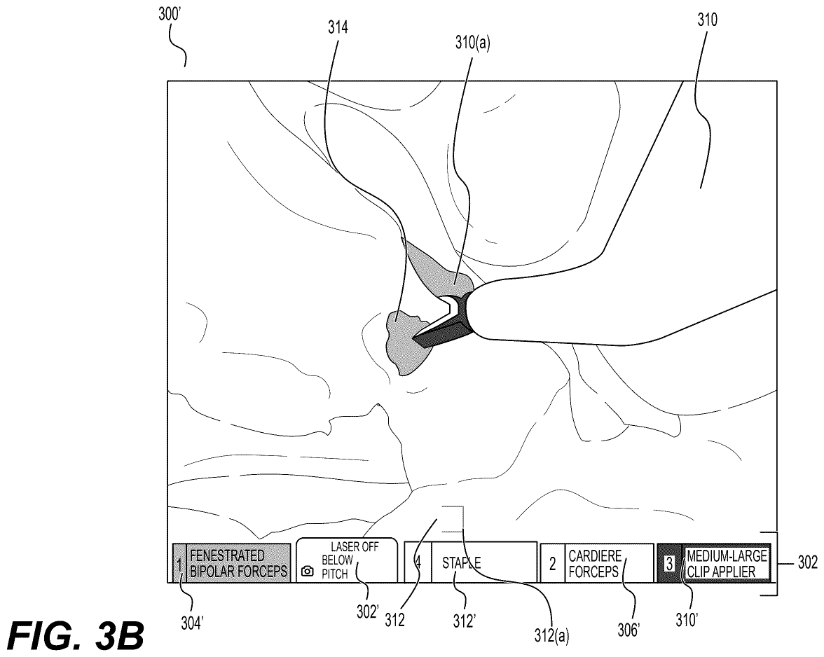

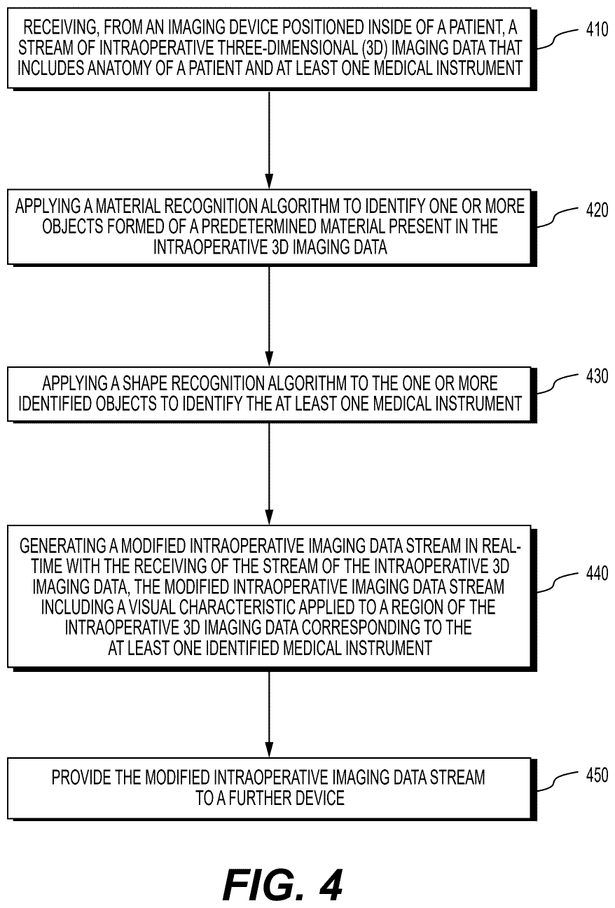

FIG. 2C illustrates an exemplary embodiment of an imaging scene 200″ similar to imaging scene 200′ of FIG. 2B. In this embodiment, the monopolar cautery instrument 208(a) is displayed in red to indicate that the instrument is in an active or “on” state. In contrast, the teal coloring of the monopolar cautery instrument 208(a) shown in FIG. 2B indicates that the instrument is in an inactive or “off” state. The laser on/off indicator 202″ is also displayed to reflect the active state of the monopolar cautery instrument 208. The color change of the monopolar cautery instrument 208(a) from teal to red provides a visual indication to the provider 130 of the operational status of the instrument, enabling quick recognition of whether the monopolar cautery instrument 208 is currently active during the medical procedure.

In some embodiments, the imaging analysis device 140 may be configured to apply different colorizations or visual characteristics to medical devices 135 based on their operational states. For example, the imaging analysis device 140 may receive tool data from actuator sensors 820 associated with the monopolar cautery instrument 208 and determine whether the instrument is in an on state or an off state. Based on the determined state, the imaging analysis device 140 may apply a first color, such as teal, to indicate an off state, and a second color, such as red, to indicate an on state. The laser on/off indicator 202″ may correspondingly update to reflect the current operational state of the instrument. This dynamic colorization based on operational state may be applied to any suitable medical device 135. The color changes may be updated in real-time, such as at least at 30 frames per second, to provide immediate feedback as the operational states of medical devices 135 change during the procedure.

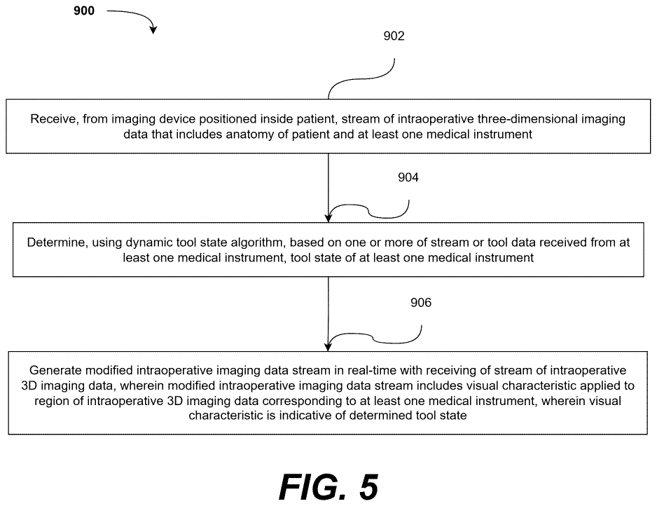

FIG. 5 is a flowchart illustrating an exemplary method 900 for intraoperative medical instrument recognition with dynamic tool state determination, according to aspects of the present disclosure. The method 900 begins at step 902, where the imaging analysis device 140 receives, from an imaging device 110 positioned inside of a patient 125, a stream of intraoperative three-dimensional (3D) imaging data that includes anatomy of the patient 125 and at least one medical device 135. At step 904, the imaging analysis device 140 determines, using a dynamic tool state algorithm, based on one or more of the stream or tool data received from the at least one medical device 135, a tool state of the at least one medical device 135. The tool state may be indicative of one or more of an open state, a closed state, or a clamped state. At step 906, the imaging analysis device 140 generates a modified intraoperative imaging data stream in real-time with the receiving of the stream of intraoperative 3D imaging data, wherein the modified intraoperative imaging data stream includes a visual characteristic applied to a region of the intraoperative 3D imaging data corresponding to the at least one medical device 135, wherein the visual characteristic is indicative of the determined tool state. The visual characteristic may include a colorization that is updated at least at 30 frames per second and may change dynamically based on changes in an output of the dynamic tool state algorithm.

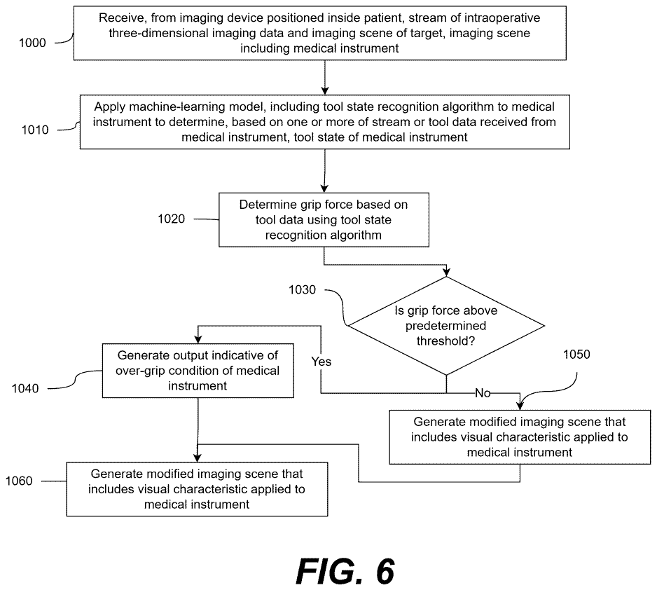

FIG. 6 is a flowchart illustrating an exemplary method for object recognition during a medical procedure with grip force monitoring and over-grip condition detection, according to aspects of the present disclosure. The method begins at step 1000, where the imaging analysis device 140 receives, from an imaging device 110 positioned inside a patient 125, a stream of intraoperative three-dimensional (3D) imaging data and an imaging scene of a target, the imaging scene including a medical device 135 and tool state data from the at least one actuator sensors 820. At step 1010, the imaging analysis device 140 applies a machine-learning model, including a tool state recognition algorithm to the medical device 135 to determine, based on one or more of the stream or tool data received from the at least one actuator sensor 820 within the medical device 135, a tool state of the medical device 135. At step 1020, the imaging analysis device 140 determines a grip force based on the tool data using the tool state recognition algorithm. At step 1030, the imaging analysis device 140 determines whether the grip force is above a predetermined threshold. If the grip force is above the predetermined threshold, the method proceeds to step 1040, where the imaging analysis device 140 generates an output indicative of an over-grip condition of the medical device 135. If the grip force is not above the predetermined threshold, the method proceeds to step 1050. At step 1060, the imaging analysis device 140 generates a modified imaging scene that includes a visual characteristic applied to the medical device 135, wherein the visual characteristic may be indicative of the tool state. The modified imaging scene may be displayed on the user device 105 or transmitted to the robotic surgery device 115 for manipulation of the medical device 135.

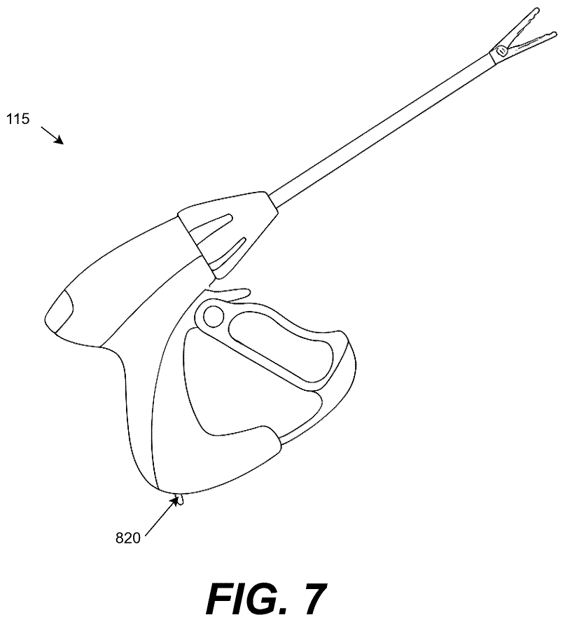

FIG. 7 illustrates a perspective view of a robotic surgery device 115 with an integrated actuator sensor 820. The actuator sensor 820 is configured to generate tool data indicative of a tool state of the robotic surgery device 115. The distal end of the elongated shaft terminates in a pair of articulated jaws or forceps, which can be opened and closed via manipulation of the trigger mechanism. The actuator sensor 820 enables monitoring of the operational state of the device, including grip force and tool state, which can be transmitted as tool data for analysis and feedback during medical procedures.

Further aspects of the machine-learning model or how it may be utilized to recognize and identify objects and characteristics thereof are discussed in further detail in the methods below.

What is claimed is

1. A computer-implemented method for intraoperative medical instrument recognition, the computer-implemented method comprising:
receiving, from an imaging device positioned inside of a patient, a stream of intraoperative three-dimensional (3D) imaging data that includes anatomy of a patient and at least one medical instrument;
determining, using a dynamic tool state algorithm, based on one or more of the stream or tool data received from the at least one medical instrument, a tool state of the at least one medical instrument; and
generating a modified intraoperative imaging data stream in real-time with the receiving of the stream of intraoperative 3D imaging data, wherein the modified intraoperative imaging data stream includes a visual characteristic applied to a region of the intraoperative 3D imaging data corresponding to the at least one medical instrument, wherein the visual characteristic is indicative of the determined tool state.

2. The computer-implemented method of claim 1, further comprising:
causing a display device to output the modified intraoperative imaging data stream in real-time with the receiving of the stream of intraoperative 3D imaging data.

3. The computer-implemented method of claim 2, further comprising:
transmitting the modified intraoperative imaging data stream to a robotic surgery device configured to manipulate the at least one medical instrument based on one or more of a position, orientation, motion, or force of the at least one medical instrument indicated by the visual characteristic applied to the intraoperative 3D imaging data.

4. The computer-implemented method of claim 1, wherein:
the one or more medical instruments include at least one actuator sensor, and
the tool data is based on a signal from the at least one actuator sensor.

5. The computer-implemented method of claim 4, wherein:
the generating of the modified intraoperative imaging data stream is based on the one or more of the tool data or the stream of intraoperative 3D imaging data over a period of time, such that the generating includes predicting one or more future position, orientation, motion, or tool state of the at least one medical instrument or a position, motion, orientation, or tool state of an occluded portion of the at least one medical instrument; and
the region where the visual characteristic is applied is based on the predicting.

6. The computer-implemented method of claim 1, wherein the visual characteristic is updated at least at 30 frames per second.

7. The computer-implemented method of claim 1, wherein:
the tool states is indicative of one or more of an open state, a closed state, a clamped state, an on state, or an off state; and
the dynamic tool state algorithm is configured to determine a grip force based on the tool data, compare the grip force to a predetermined threshold, and in response to the grip force being above the predetermined threshold, generate an output indicative of an over-grip condition of the medical instrument.

8. The computer-implemented method of claim 1, wherein the visual characteristic includes a colorization.

9. The computer-implemented method of claim 8, wherein the colorization is configured to change based on changes in an output of the dynamic tool state algorithm.

10. The computer-implemented method of claim 4, wherein the dynamic tool state algorithm has been trained based on a training stream or training tool data from at least one training actuator sensors and medical instrument labels assigned to one or more of portions of the training stream or the training data from the at least one training actuator sensors, to predict a likelihood that a particular signal from the at least one actuator sensor corresponds to a particular tool state.

11. A computer-implemented method for object recognition during a medical procedure, comprising:
receiving, from an imaging device positioned inside a patient, a stream of intraoperative three-dimensional (3D) imaging data and an imaging scene of a target, the imaging scene including a medical instrument;
applying a machine-learning model, including a tool state recognition algorithm to the medical instrument to determine, based on one or more of the stream or tool data received from the medical instrument, a tool state of the medical instrument; and
generating a modified imaging scene that includes a visual characteristic applied to the medical instrument.

12. The computer-implemented method of claim 11, wherein the medical instrument contains one or more actuator sensors.

13. The computer-implemented method of claim 12, wherein the one or more actuator sensors are configured to provide tool data to the tool state recognition algorithm.

14. The computer-implemented method of claim 13, wherein the machine-learning model has been trained based on a training stream of intraoperative three-dimensional (3D) imaging data or training tool data from at least one training actuator sensors and medical instrument labels attached to one or more of portions of the training stream or the training tool data from the at least one actuator sensors, to predict a likelihood that a particular signal from the at least one actuator sensor corresponds to a particular tool state.

15. The computer-implemented method of claim 11, wherein the visual characteristic includes a colorization that changes dynamically in response to a detected change by the tool state recognition algorithm.

16. The computer-implemented method of claim 14, wherein the tool state recognition algorithm is configured to determine a grip force based on the tool data, compare the grip force to a predetermined threshold, and in response to the grip force being above the predetermined threshold, generate an output indicative of an over-grip condition of the medical instrument.

17. The computer-implemented method of claim 15, wherein a change in the colorization is proportional to a detected change in the tool state.

18. The computer-implemented method of claim 11, wherein the visual characteristics is only to applied to a portion of the stream including the medical instrument.

19. The computer-implemented method of claim 11, wherein the machine-learning model is configured to use one or more of the stream or the tool data to auto calibrate in real-time.

20. A system for an intraoperative instrument recognition, comprising:
at least one medical instrument that includes at least one actuator sensor configured to generate tool data indicative of a tool state of the at least one medical instrument;
at least one imaging device configured to capture a stream of intraoperative three-dimensional (3D) imaging data; and
an imaging analysis device that includes:
at least one memory storage:
instructions for intraoperative medical instrument recognition, including:
a dynamic tool state recognition algorithm configured to recognize tool states based on one or more of the stream or the tool data from the at least one actuator sensor;
a material recognition algorithm; and
a shape recognition algorithm; and
at least one processor operatively connected to the at least one memory storage and the at least one medical instrument, and configured to execute the instructions to perform operations including:
receiving, from the imaging device, the stream of intraoperative three-dimensional (3D) imaging data from inside of a patient, the stream including anatomy of the patient and the at least one medical instrument;
applying the material recognition algorithm to identify one or more objects formed of a predetermined material present in the intraoperative 3D imaging data;
applying the shape recognition algorithm to the one or more identified objects to identify the at least one medical instrument;
applying the dynamic tool state algorithm to the at least one identified medical instrument to identify a tool state of the at least one medical instrument; and
generating a modified intraoperative imaging data stream in real-time with the receiving of the stream of intraoperative 3D imaging data, wherein the modified intraoperative imaging data stream includes a visual characteristic applied to a region of the intraoperative 3D imaging data corresponding to the at least one identified medical instrument, wherein the visual characteristic is indicative of the identified tool state.
